(19) United States
(12) Patent Application Publication
(10) Pub. No.: US 2024/0023838 A1
(43) Pub. Date: Jan. 25, 2024

(54) NON-INVASIVE BLOOD GLUCOSE MONITORING SYSTEM

(71) Applicant: Kennesaw State University Research And Service Foundation, Inc., Kennesaw, GA (US)

(72) Inventor: Maria Valero de Clemente, Marietta, GA (US)

(21) Appl. No.: 18/354,426

(22) Filed: Jul. 18, 2023

Related U.S. Application Data
(60) Provisional application No. 63/390,336, filed on Jul. 19, 2022.

(51) Int. Cl. A61B 5/145; A61B 5/1455; G16H 40/67

(52) U.S. Cl. CPC A61B 5/14532; A61B 5/1455; G16H 40/67; A61B 5/6826

(57) ABSTRACT

A non-invasive optical glucose monitoring (NIO-GM) system for continuous monitoring of blood glucose levels in a user. The system uses infrared spectroscopy through use of a laser and camera in a main body to collect data. Data is then pre-processed and subsequently analyzed with a neural network, which provides blood glucose level estimations. Estimations are analyzed for accuracy.

### CROSS-REFERENCE TO RELATED APPLICATION

This application is a U.S. Nonprovisional of and claims the benefit of U.S. Provisional Application No. 63/390,336 filed Jul. 19, 2022, which is hereby incorporated by reference in its entirety.

### TECHNICAL FIELD

This disclosure generally relates to the field of non-invasive blood glucose monitoring in combination with machine learning.

### BACKGROUND

Diabetes mellitus is a disease characterized by high blood glucose levels resulting from dysregulation of the hormone insulin. Diabetes is managed through physical activity and dietary modification and requires careful monitoring of blood-glucose concentration. Diabetes affects approximately one out of every 10 people in the United States (USAHealth. “About Diabetes Care at USA Health” (2022)). Its prevalence has increased from 23.4 million Americans in 2015 to 30.3 million in 2021 and continues to rise at an alarming rate (CDC, “National Diabetes Statistics Report”, (2022)).

The standard technique for determining blood glucose concentration involves using a glucometer (Salacinski, A. J., et al., J. of Diabetes Sci. and Tech. 95-99 (2014)). In brief, this device determines glucose concentration either in droplets of blood from a finger prick in portable devices or with a laboratory blood draw in a clinical setting. Such techniques may be invasive, painful, uncomfortable, and otherwise disadvantageous for health. Successful management of diabetes involves monitoring blood glucose levels multiple times per day using this glucometer. Taking repeated finger pricks over the course of a day is painful and creates a risk of infection at the collection site (Farage, M. A., et al., American J. of Clinical Derm., 73-86 (2009)). The situation is especially burdensome in the aging population, where skin elasticity is reduced, and the immune response is slow. Glucose monitoring in diabetes may also be performed by pervasive devices with the capacity of in situ computation. J. Hartz, et al., Current cardiology reports, 18(12): 1-11 (2016). Some methods involve implanting a thin lancelet subcutaneously. This provides continuous monitoring. An inherent issue of this minimally invasive method is the risk it poses for tissue damage and infection. C. F. So, et al., Medical Devices (Auckland, NZ), 5: 45 (2012).

According to the current literature (Shang, T., et al., J. of diabetes science and technology, 168-214 (2022)), there are several types of non-invasive glucose monitoring systems including thermal, electrical, nanotechnology, and optical systems. Non-invasive optical glucose monitoring (NIO-GM) systems are based on optical glucose monitoring. Non-invasive fluid sampling (NIFS-GM) systems are based on fluid sample glucose estimation. Minimally invasive devices (MI-GM) involve the insertion of a sensor into the subcutaneous tissue underneath the skin. FIGS. 1A-1C illustrate an example of each type of non-invasive and minimally invasive blood glucose monitoring.

These noninvasive approaches to monitoring blood glucose have limitations. Electrical approaches are sensitive to temperature and may lack sources capable of producing meaningful amounts of energy that can penetrate the tissue for noninvasive analysis. W. V. Gonzales, et al., Sensors, 19(4): 800 (2019); A. Tura, et al., Sensors, 10(6): 5346-5358 (2010); M. Gourzi, et al., Journal of Med. Eng. & Tech., 29(1): 22-26 (2005); H. Melikyan, et al., Medical Eng. & Phys., 34(3): 299-304 (2012); P. H. Siegel, IEEE, 1-3 (2015); S. A. Weinzimer, Diabetes Tech. & Thera., 6(4): 442-444 (2004). Thermal approaches are susceptible to interference from environmental conditions and may be sensitive to sweat. C. D. Malchoff, et al., Diabetes Care, 25(12): 2268-2275 (2002); J. M. Buchert, Optical Security and Safety, 5566.100-111 (2004); O. K. Cho, et al., Clinical Chem., 50(10): 1894-1898 (2004); F. Tang, et al., Sensors, 8(5): 3335-3344 (2008); Y. Tanaka, et al., Photons Plus Ultrasound: Imaging and Sensing, 10494. SPIE, 494-498 (2018). Thermal approaches are also expensive and have long integration times. W. V. Gonzales, et al., Sensors, 19(4): 800 (2019). Nanotechnology approaches, potentially in a combination with optical resources, have potential toxicity issues, short lifespans, limitations associated with photostability, and high costs. P. W. Barone, et al., Analytical Chem., 77(23): 7556-7562 (2005); J. C. Pickup, et al., Biosensors and Bioelectronics, 20(12): 2555-2565 (2005); D. C. Klonoff, J. of Diabetes Sci. and Tech., 6(6): 1242-1250 (2012); P. W. Barone et al., J. of Diabetes Sci. and Tech., 3(2): 242-252 (2009); L. Chen, et al., Sensors, 18(5): 1440 (2018).

Optical technologies include Mid-Infrared Spectroscopy, which is a vibrational spectroscopy technique. J. Coates, Applied Spec. Rev., 33(4): 267-425 (1998).; S. Liakat, et al., Biomedical Opt. Exp., 4(7): 1083-1090 (2013); S. Liakat, et al., Biomedical Opt. Exp., 5(7): 2397-2404 (2014). Optical technologies may be expensive and limited due to light penetration depths. Raman Spectroscopy provides a way to measure molecular compositions through inelastic scattering but may be prone to interference by other molecules such as hemoglobin. R. Pandey, et al., Accounts of Chem. Res., 50(2): 264-272 (2017); Y. Xu, et al., Biomed. Sensing, Imaging, And Tracking Tech., 2976. SPIE, 10-19 (1997); S. M. Lundsgaard-Nielsen, PloS one, 13(5): e0197134 (2018). Raman Spectroscopy may also include a long collection time. Far-Infrared Spectroscopy may have less scattering of mid-infrared approaches but may also have strong water absorption that makes the identification of molecules in the sample difficult. F. Tang, et al., Sensors, 8(5): 3335-3344 (2008). Time of Flight and Terahertz Time-Domain Spectroscopies use short and ultrashort laser pulses to measure travel time of reflected signals. S. Gusev, et al., 2017 Progress. In Electromagnetics Research Symposium Spring (PIERS). IEEE, 3229-3232 (2017); O. Cherkasova, et al., Optical and Quantum Electronics, 48(3): 1-12 (2016). They may have long measurement times and low spatial and depth resolutions.

Near-Infrared Spectroscopy (NIR), which relies on absorption and scattering of wavelengths, may be low cost. NIR has a signal intensity that is directly proportional to the concentration of the analyte, requires minimal sample preparation, and works in the presence of interfering substances such as glass and plastic. Monte-Moreno et. al used this method to estimate blood sugar, obtaining a Clarke error grid placed 87.7% of points in zone A, 10.3% in zone B, and 1.9% in zone C. E. Monte-Moreno, Artificial Int. in Med., 53(2): 127-138 (2011). Yamakoshi et al. also used NIR for estimating glucose, obtaining a Clarke error grid placed 90.05% of points in zone A, and 9.95% in zone B. K. Yamakoshi, J. of Biomed. Opt., 11(5): 054028 (2006). Also, Alarcon-Paredes et al. used this technology to estimate blood sugar with a Clarke error placed of points in zone A, and 9.68% in zone B. A. Alarcon-Paredes, Applied Sci., 9(15): 3046 (2019).

Therefore, there exists a need for a monitoring system that allows someone suffering from diabetes to monitor their blood sugar in a better way.

### SUMMARY

It is to be understood that this summary is not an extensive overview of the disclosure. This summary is exemplary and not restrictive and it is intended to neither identify key or critical elements of the disclosure nor delineate the scope thereof. The sole purpose of this summary is to explain and exemplify certain concepts of the disclosure as an introduction to the following complete and extensive detailed description.

The present disclosure relates to a blood glucose monitoring system. The system can be a non-invasive optical glucose monitoring (NIO-GM) system.

The present disclosure relates to a main body configured to be positioned on a portion of a user. Such a main body can further comprise one or more sensors disposed within the main body and configured to collect information relating to user characteristics from the portion of the user. Sensors may include but are not limited to small, intelligent glucose sensors. As a non-limiting example, a main body includes but is not limited to a clip, as shown in FIGS. 5A-5C. As a non-limiting example, user characteristics may include but are not limited to blood glucose level estimations. The portion of the user includes but is not limited to an ear or a finger. The main body includes a first portion and a second portion. The first portion includes a laser, and the second portion includes a camera. The main body collects information from the portion of the user by shining the laser in the first portion through a portion of the user. Shining the laser produces an image that is captured by the camera through infrared spectroscopy. The collected information is then pre-processed before being sent to a neural network. Information includes but is not limited to data and raw data pertaining to characteristics of a user. Such characteristics include but are not limited to blood glucose levels, blood glucose level estimations, glucose levels, glucose level estimations, and the like.

The present disclosure relates to a computing device. A computing device is functionally disposed to allow operations of a model. A model may include but is not limited to any neural network or other machine learning enabled function. Such a neural network can be selected from a group consisting of but not limited to a convolutional neural network (CNN) and an Artificial Neural Network (ANN). The neural network receives the data collected by the main body as input. The neural network uses data collected by the main body to first be trained. About 80% of the collected data is used for training, and about 20% of the collected data is used for testing. The neural network then analyzes the data to produce a blood glucose level estimation of the user.

The present disclosure relates to a cloud-based database configured to store the glucose level estimation from the neural network. The cloud-based database allows for real-time results for a user.

The present disclosure relates to a mobile application coupled to the cloud-based database to display real-time glucose level estimations to a user. The mobile application provides continuous glucose monitoring and history data for users. The mobile application also allows users to manually enter glucometer readings for comparison purposes and for additional training of the neural network.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIGS. 1A-1C display three types of non-invasive glucose monitoring systems—non-invasive optical glucose monitoring (FIG. 1A), minimally invasive glucose monitoring (FIG. 1B), and non-invasive fluid sampling glucose monitoring (FIG. 1C).

FIG. 2 displays an example of a non-invasive monitoring system according to the present disclosure.

FIG. 3 displays an example of Near-Infrared Spectroscopy (NIR).

FIG. 4 displays a process flow according to the present disclosure.

FIGS. 5A-5C display a main body according to the present disclosure.

FIG. 6 displays an Artificial Neural Network (ANN) model according to the present disclosure.

FIG. 7 displays an example of a VGG16 architecture according to the present disclosure.

FIG. 8 displays a workflow according to the present disclosure.

FIGS. 9A-9B show images collected from fingertips (FIG. 9A) and ear lobes (FIG. 9B) of volunteers.

FIGS. 10A-10B display confusion matrices for finger readings (FIG. 10A) and ear readings (FIG. 10B) according to the present disclosure.

FIG. 11 displays a device and external communication with other devices according to the present disclosure.

FIG. 12 displays an example of architectural integration of systems and devices according to the present disclosure with pervasive devices.

FIG. 13 displays a mobile application according to the present disclosure.

FIG. 14 displays a manual operation of a mobile application according to the present disclosure.

FIG. 15 displays a mobile application according to the present disclosure.

FIG. 16 displays an example of finger spectroscopy images collected from one individual.

FIG. 17 displays an example of data collection according to the present disclosure.

FIG. 18 displays a glucometer.

FIG. 19 displays an example of an image tensor conversion according to the present disclosure.

FIG. 20 displays a histogram of Red Blue Green (RGB) Intensity Values in an image.

FIG. 21 displays an example of a demonstration of a measurement dataset creation.

FIG. 22 displays a Clarke Error Grid.

FIG. 23 displays a Parkes Error grid.

FIG. 24 displays an example of a Clarke Error Grid of an AdaBoost model with KNeighbors Trained on a Red-Intensity Dataset.

FIG. 25 displays an example of a Parkes Error grid.

FIG. 26 displays an example of a Clarke error accuracy comparison with other research approaches.

FIG. 27 displays a ranking of model averages of mean absolute errors (MAEs) of AdaBoost, KNeighbors, Random Forest, XGBoost, HGB, Support Vector, Bayesian Ridge, Elastic Net, MobileNetV2, and VGG16 models.

FIG. 28 displays a ranking of dataset averages of mean absolute errors (MAEs) of ranked RGB Intensity, Red Measurement, Red Intensity, RGB Measurement, Green Measurement, Blue Intensity, Blue Measurement, Green Intensity, Measurement, and Image Tensor datasets.

FIG. 29 displays a comparison of average error based on gender.

FIG. 30 displays a comparison of average error based on age.

FIG. 31 displays a comparison of average error based on race and ethnicity.

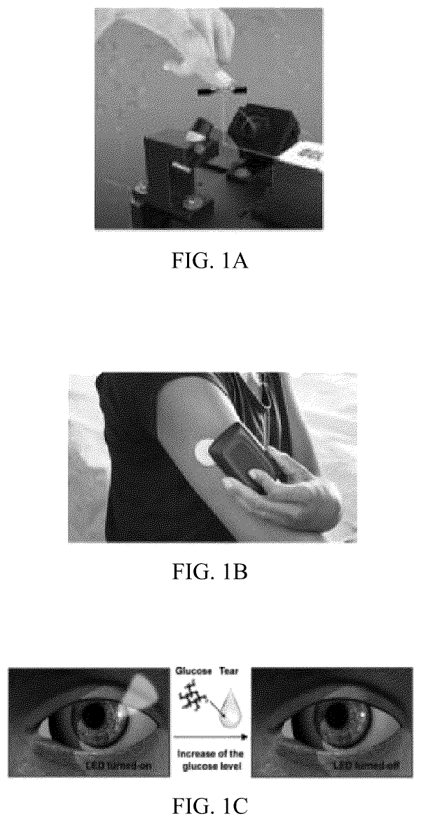

### DETAILED DESCRIPTION

The present disclosure can be understood more readily by reference to the following detailed description, examples, drawings, and claims, and their previous and following description. However, before the present compositions, systems, and/or methods are disclosed and described, it is to be understood that this disclosure is not limited to the specific devices, systems, and/or methods disclosed unless otherwise specified, as such can, of course, vary. It is also to be understood that the terminology used herein is for the purpose of describing particular aspects only and is not intended to be limiting.

### I. Definitions

It should be appreciated that this disclosure is not limited to the compositions and methods described herein. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

Unless defined otherwise, all composition percentage values used herein are given in terms of weight percentage.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, the Beer-Lambert Law of Absorption is a law associated with light absorption that is governed by the below equation.

I=I₀10^{(-l,∈,c)}=I₀10^{(-l,μ}^{_{α}}⁾

where I₀ is the initial light intensity (W/cm²), I is the intensity of the ith at any depth within the absorption medium in W/cm², 1 is the absorption depth within the medium in cm, ε is the molar extinction coefficient in L/(mmol cm), and c is the concentration of absorbing molecules in mmol/L. The product of c and ε is proportional to the absorption coefficient (μₐ).

II. Non-Invasive Blood Glucose Monitoring System

The present disclosure relates to a non-invasive, reliable, and user friendly glucose monitoring system. The system offers significant improvements in accuracy, accessibility, and usability compared to existing techniques. Accessibility may be enhanced by a reduced number of components required for some embodiments described herein. In a non-limiting example, some devices may require only a laser light, a camera, and a processing system.

Systems disclosed herein are capable of being used with medical diagnostics, spectroscopy-based techniques, machine learning, and/or image processing. In a particular example, spectroscopy, image processing, and machine learning techniques may be integrated into the monitoring system to achieve accurate, non-invasive blood glucose estimation. Advanced algorithms and spectroscopic image analysis allow devices described herein to overcome limitations of existing techniques, such as inaccuracies, inconsistency, and invasiveness. Such systems described herein can estimate glucose levels and increase life quality for people with diabetes. Such a system eliminates needs for frequent invasive procedures, empowers individuals to monitor their glucose levels conveniently, and facilitates timely interventions for better disease management.

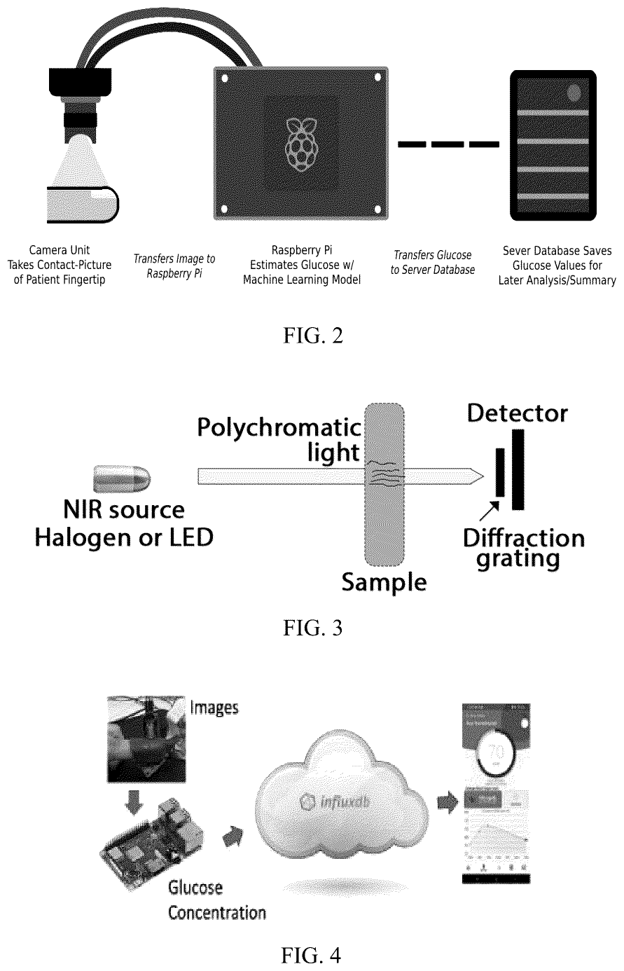

The present disclosure relates to a non-invasive optical glucose monitoring (NIO-GM) system. The system utilizes laser technology and spectroscopy images to accurately estimate and monitor blood glucose levels. Such laser technology may include a light beam. The light beam is directed at a human tissue. Once the light beam has contacted the human tissue, the energy absorption, reflection, or scattering is used to estimate glucose concentration (Pitzer, K. R., et al., Clinical Diabetology 307-314 (2001)). The system of the present disclosure is portable and inexpensive. The present disclosure additionally includes novel features such as incorporating a machine learning statistical approach that has not been used before for glucose estimation. The present disclosure additionally relates to a light method that can be run in pervasive devices presents results in real-time. An Example of a non-invasive system is shown in FIG. 2. This non-limiting example image includes at least a camera, a Raspberry Pi, and a server database. The present disclosure additionally relates to producing results with more rigorous evaluations, including Parkes error grid evaluations. Parkes error grids include a more robust method to evaluate glucose estimation devices. A. Pfutzner, et al., J. of Diabetes Sci. and Tech., 7(5): 1275-1281 (2013).

Optical glucose monitoring systems may utilize various optical methodologies. Such optical methodologies include but are not limited to fluorescence spectroscopy (Pickup, J. C., et al., J. of diabetes science and technology, 62-71 (2013)), Raman spectroscopy (Enejder, A. M. K., et al., J. of biomedical optics 031114 (2005)), photoacoustic spectroscopy (Pai, P. P., et al., IEEE Transactions on Circuits and Systems I: Regular Papers, 663-676(2017)), optical coherence tomography (Haxha, S., et al., IEEE Photonics Journal 1-11 (2016)), occlusion spectroscopy (Amir, O., et al., “Continuous noninvasive glucose monitoring technology based on occlusion spec-troscopy” (2007)), and near-infrared absorption spectroscopy (Robinson, M. R., et al., Clinical Chemistry 1618-1622 (1992); Alarcon-Paredes, A., et al., Applied Sciences 3046 (2019); Kasahara, R., et al., Biomedical optics express, 289-302 (2018); Rachim, V. P., et al., Sensors and Actuators B: Chemical 173-180 (2019); Maruo, K., et al., IEEE Journal of selected topics in quantum electronics, 322-330(2003)).

As a non-limiting example, near-infrared absorption spectroscopy (NIR) provides advantages due to its low cost and practicality. The present disclosure relates to the use of near-infrared absorption spectroscopy, which is based on the Beer-Lambert Law of Absorption as understood in the art. In NIR, a polychromatic light source (Light Emitting Diode (LED)) is radiated through the sample. A diffraction grating then splits the transmitted radiation into its constituent wavelengths to a camera (sensor) and the images are analyzed by a computer board (detector). FIG. 3 illustrates this absorption mode. The present disclosure uses image capture via camera(s) for superior speed, replicability, and accessibility, in comparison to other forms of spectroscopy measurement, such as light intensity and Photoplethysmography (PPG) signals. The Beer-Lambert Law of Absorption provides a concentration of absorbing molecules is based on the associated equation. According to the present disclosure, the effect of other blood components and absorbing tissue components affect the amount of light absorbed. As a result, the total absorption coefficient is the summation of the absorption coefficients of all the absorbing components (Jacques, S. L., Physics in Medicine & Biology R37 (2013)). To minimize the absorption due to all the other components, the wavelength of the light source is chosen so that the light source is highly absorbed by glucose and is mostly transparent to blood and tissue components. This allows the near-infrared absorption spectroscopy of the present disclosure to more accurately obtain data relating to blood glucose levels. The present disclosure relates to the use of near-infrared absorption spectroscopy in combination with a NIO-GM system.

The present disclosure relates to computing power of sensors and Internet of Things (IoT) devices. The present disclosure relates to a combination of computing power of sensors and IoT devices with NIO-GM systems to continuously analyze blood glucose from a microcomputer and a sensor embedded within a main body positioned on the finger or ear. Images of the rotational and vibrational transitions of chemical bonds within the glucose molecule are created using infrared spectroscopy, and incident light reflection is used to measure the corresponding fluctuation. The images are converted into an array list, which is used to provide entries to an Artificial Neural Network (ANN) to create an estimate of blood glucose concentration. The system is easy to use and is paired with a mobile application for free-living environments. FIG. 4 shows an overview of the system.

The present disclosure relates to IoT (Internet of Things) technologies. IoT technologies leverage power computing and low energy consumption of sensor devices and a Raspberry Pi camera for building the glucose-monitoring prototype (Raspberry Pi, “Raspberry pi 4 model B specifications”, (2021)). A Raspberry Pi camera is capable of capturing a set of images where a visible light laser passes through skin tissue. Glucose concentration can then be estimated by an artificial neural network model using the absorption and scattering of light in the skin tissue. Various programming languages can be used in conjunction with the glucose estimation including but not limited to TensorFlow, Keras, and Python code. While the Raspberry Pi camera captures images, a laser light captures absorption. The specifications of the laser light can be found in Table 1.

TABLE 1
Light Laser Specifications

| Brand | Icstation | |
|---|---|---|
| Model Number | KY-008 5 mW Red Laser Transmitter | |
| Module | Infrared | |
| Part Number | 276810 | |
| Working Voltage | 5 | mW |
| Wavelength | >650 | nm |

| | |
|---|---|
| Size | 24 × 15 mm/0.94 × 0.59 inch(L*W) |

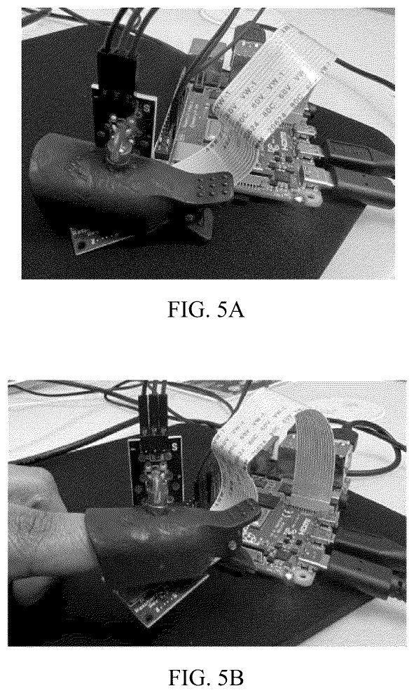

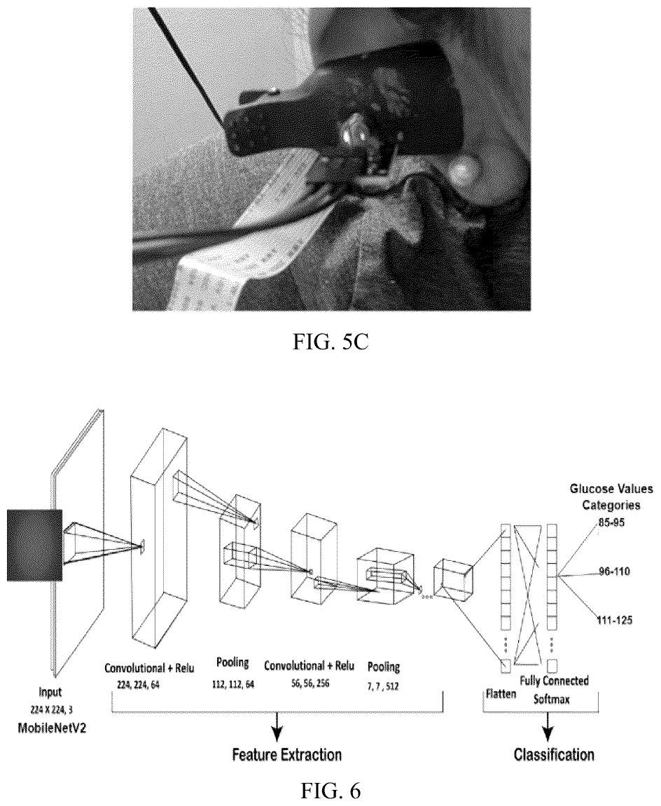

The present disclosure relates to a main body of a device that can be positioned on a portion of a user. Such a main body includes a compact and portable spectroscopy module integrated with advanced image processing and machine learning algorithms. A portion of a user may include but is not limited to a finger or earlobe. The optional choice to position a device on an earlobe of a user is unique and may allow for embodiments of the device as an earring or other jewelry. Such an application may allow a user to constantly wear the device for continued glucose monitoring. The main body of a device may capture spectroscopic images of the target area, such as the forearm or fingertip, using the device's sensors. These images contain valuable information about glucose levels within the tissues. This main body includes a laser on its first portion and a camera on its second portion, as shown in FIGS. 5A-5C. The camera can be an RPi camera. The camera captures a number of images during operation. This number of images can be large. The quality of images and the accuracy of absorption spectroscopy can be affected by a number of factors. Such factors include but are not limited to skin pigmentation, skin thickness, skin roughness, moving and breathing of a subject, ambient temperature, formability of a device's enclosure to a subject's finger or ear, and the like. Additionally, databases with more data points improve the robustness of artificial neural network models.

Once the spectroscopy images are obtained, the device applies image processing techniques to extract relevant features, such as image tensors, color intensity, and statistical image information. These features are then fed into a machine learning model that has been trained on a large dataset of spectroscopy images and corresponding glucose measurements.

The present disclosure relates to models. The present disclosure relates to machine learning models and methodologies. The present disclosure further relates to a combination of machine learning models and methodologies with NIO-GM systems to monitor blood glucose levels in real time. The machine learning model utilizes its learned knowledge to accurately estimate the glucose levels from spectroscopy images. The estimation is based on the correlation between the extracted image features and glucose concentrations. The device provides real-time glucose readings, displaying them on a user-friendly interface for easy interpretation.

The present disclosure may relate additionally to a head model, which sits on top of a base model. The present disclosure additionally relates to an activation layer, which uses the Rectified Linear Unit (ReLu) activation function (Ramachandran, P., et al., arXiv preprint, arXiv:1710.05941 (2017)). The ReLU is a piecewise linear function that will output the input directly if it is positive; otherwise, it will output zero. The present disclosure additionally relates to a pooling layer, which incorporates feature-down sampling. It is applied to each layer in the three-dimensional volume. The present disclosure additionally relates to a fully connected layer, which involves flattening. The entire pooling feature map matrix is transformed into a single column, which is then supplied to the neural network for processing. These attributes are put together to make a model using the fully linked layers. Finally, the output is classified using a ‘Softmax’ activation function. The ANN model was trained using the ADAM technique, which included a total of 20 epochs, a batch size of 1, an initial learning rate of 1e-4, and a 0.5 dropout was considered. The next step was to train and test the model. About 80% of the data was used for training the model, and about 20% was used for testing the model. FIG. 6 shows the ANN used for our glucose estimation process. For evaluation purposes, classification reports, accuracy, and a confusion matrix may be used to assess the accuracy of the model.

Due to the large number of images, a convolutional neural network (CNN/ConvNet) approach can properly be used. The large number of images required to continuously monitor blood glucose levels daily provides a large database to train and analyze neural networks. The convolutional layer is the first layer of a CNN network and is the main building block that handles most of the computational work. A number of libraries, such as VGG16, Tensor Flow, Keras, MobileNetV2, Matplotlib, Numpy, and the like can be used. Sandler, M., et al., Proceedings of the IEEE conference on computer vision and pattern recognition, 4510-4520 (2018); Tammina, S., Int'l J. of Sci. and Res. Pub., 9(10): 143-150 (2019). CNN models pass filters through images (represented as tensors) to extract features such as edges, shapes, and colors. These two-dimensional features are then flattened and mapped as scalar data, which is then processed through normal neural network layers. Albawi, S., 2017 Int'l Conf. on Eng. and Tech., 1-6 (2017). CNN models can use different types of filters for images of varying sizes, providing a wide range of applications.

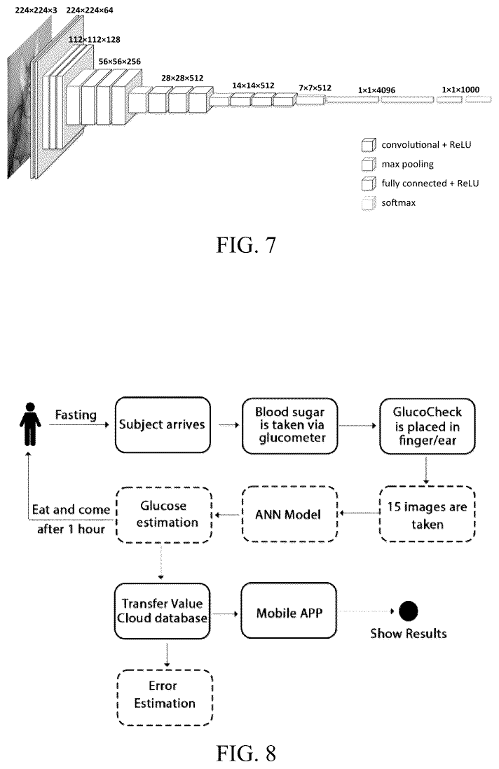

One, non-limiting example of a CNN is VGG16, as mentioned above. VGG-16 is a 16 layered deep CNN. A pre-trained version of the network can be loaded. Some pre-trained versions are already trained on more than a million images from an ImageNet database. Russakovsky, O., et al., Int'l J. of Comp. Vision, 115(3): 211-252 (2015). A pre-trained network can classify images into about 1000 object categories. Such networks learn rich feature representations for a wide range of images. Such models can also be changed to output a single numeric value (blood glucose), instead of the 1000 categories a model was trained on. The network may have an image input size of 224-by-224. This size can be changed to fit 160-by-120 and other sizes as needed. VGG16 has the ability to detect many different features and patterns and may perform better in some aspects compared to other models. Tammina, S., Int'l J. of Sci. and Res. Pub., 9(10): 143-150 (2019). An example of the VGG-16 architecture can be found in FIG. 7 (Taken from Shi, B., et al., SPIE, 98 (2018)).

An additional, non-limiting example of a CNN includes MobileNetV2, as discussed herein. MobileNetV2 is a mobile architecture that enhances the performance of mobile models across various model sizes, tasks, and benchmarks. In contrast to conventional residual models, which use expanded representations for the input, the MobileNetV2 architecture uses an inverted residual structure. An inverted residual structure includes inputs and outputs of the residual block that are thin bottleneck layers. Such model performs well considering its computational power. Therefore, MobileNetV2 is capable of being trained normally without using a pre-trained model version. MobileNetV2 includes numerous advantages including but not limited to low computational power usage, fast training times, and high-performance. Sandler, M., et al., Proceedings of the IEEE conference on computer vision and pattern recognition, 4510-4520 (2018). Additionally, MobileNet-v2 is a lightweight, 53-layer deep CNN method used to improve the classification of images with a limited dataset.

Additionally, linear models can be used instead of CNN models. Linear models perform machine learning and statistical modeling. Linear models additionally are capable of using a large number of algorithms for function approximation, decision making, regression, classification, clustering, prediction, and the like. Like CNNs, linear models are also capable of a wide range of applications and display enhanced performance. Linear models may be faster and less computationally intensive compared to neural networks and may be capable of providing similar or better results than neural networks in certain instances. The present disclosure may relate to linear models applying bagging, boosting, or ensemble learning techniques. Such techniques allow for higher performance, lower error, and more optimized training. A mix of models using these techniques may be used to determine the most effective for estimating blood glucose.

Linear models include but are not limited to Random Forest, Support Vector Machine, Bayesian Ridge, XGBoost, AdaBoost Ensemble, Histogram Gradient Boosting, Elastic Net, and KNeighbors. Donges, N., Contributor, E., entrepreneur, N.a.: Random forest algorithm: A complete guide; Raj, A.: Unlocking the true power of support vector regression (2020); Rothman, A.: The bayesian paradigm amp; ridge regression (2020); Sklearn.ensemble.adaboostregressor, https://scikit-learn.org; Brownlee, J.: Histogram-based gradient boosting ensembles in python (2021); Verma, Y.: Hands-on tutorial on elasticnet regression (2021); Brownlee, J.: Xgboost for regression (2021). To apply these methods to the spectroscopic images, data transformation techniques may be required to create new suitable databases for each method, as discussed herein. CNN and linear models may offer different advantages. CNN models can be used on tensor data because the algorithms are based on Linear Algebra suitable for use with multi-dimensional matrices (tensors). Linear models are suitable for all scalar data and use a wide variety of statistical techniques to approximate the function of the data.

As a non-limiting example of a linear model, random forest regressor (RFR) may be used. RFR is a supervised learning algorithm built on Decision Trees and the Ensemble Learning Approach. Dong, X., et al., Frontiers of Comp. Sci., 14(2): 241-258 (2020). Decision Trees are tree-diagrams of statistical decisions that lead users to a specific outcome, result, or prediction. Random Forest uses an optimized approach to ensemble learning called bagging (bootstrap-aggregating). Bagging creates multiple decision trees that train on random segments of the training data. These trees are then used in unison to predict unknown values. RFR's combination of Decision Trees and bagging in addition to its high performance in many domains may prove advantageous. Donges, N., Contributor, E., entrepreneur, N.a.: Random forest algorithm: A complete guide., https://builtin.com/data-science/random-forest-algorithm.

As an additional, non-limiting example of a linear model, support vector regressor (SVR) may be used. SVR works on the principle of the Support Vector Machine (SVM). Raj, A.: Unlocking the true power of support vector regression (2020); Noble, W. S., Nature Biotech., 24(12): 1565-1567 (2006). This model uses simple regression algorithms to fit a line, curve, and/or plane through data to create an approximate function. In simple regression, a goal is to minimize the error rate while in SVR it is to fit the error inside a certain threshold. The flexibility of SVR allows a user to decide how much error is acceptable in the model. SVR will then find an appropriate line (or curve or plane) to fit the data accordingly. This technique may be advantageous due to its ability to reduce overfitting and handle outliers in data. SVR is a well-performing and versatile model.

As an additional, non-limiting example of a linear model, Bayesian ridge regressor (BRR) may be used. Ridge Regression is a classical regularization technique used in Statistics and ML. Rothman, A.: The bayesian paradigm amp; ridge regression (2020). Bayesian regression allows a natural mechanism to survive insufficient or less distributed data by generalizing the data. Generalizing is capable of significantly reducing overfitting and handling outliers. In addition, this model is capable of outputting a probability distribution. A probability distribution allows output of multiple predicted values where the model chooses the most likely value. BRR may be advantageous due to its ability to perform well regardless of data quality.

As an additional, non-limiting example of a linear model, XGBoost Regressor (XGB) may be used. XGB uses gradient boosting, an ensemble learning technique similar in some aspects to Random Forest. XGB trains multiple decision trees to create an ensemble learner and uses intuition that the best possible next model, combined with previous model(s), minimizes the overall prediction error. Through combining multiple models training, XGB is capable of achieving high performance. XGB effectively deals with insufficient data and outliers. Extreme Gradient Boosting (XGBoost) is an efficient, open-source implementation of this gradient boosting algorithm. XGBoost allows for increased training speed and model performance.

As an additional, non-limiting example of a linear model, a Histogram Gradient Boosting Regressor (HGB) may be used. Histogram-based gradient boosting is an algorithm that uses a similar gradient boosting compared to XGBoost. HGB employs binning, instead of outputting a single value for blood glucose as with XGBoost. Binning converts continuous values into categories, similar to those used in classification scenarios. Brownlee, J.: Histogram-based gradient boosting ensembles in python (2021). By converting regression values to classification values, training speed is increased, and the amount of memory used is reduced. Such benefits may make HGB faster and lighter compared to the XGBoost algorithm.

As an additional, non-limiting example of a linear model, an AdaBoost Ensemble Regressor (ABR) may be used. An AdaBoost regressor is a metaestimator that begins by fitting another model on the original dataset. An ABR then fits additional copies of that model on the same dataset. While fitting additional copies, the weights of instances are adjusted according to the error of the current prediction. Sklearn.ensemble.adaboostregressor, https://scikit-learn.org. ABR creates more versions of the same model to tackle different sections of the training data, reducing error overall. Due to the large number of varying estimators that AdaBoost creates, the model is less prone to overfitting than other models. The present disclosure relates to AdaBoost trained with the KNeighbors model, as described herein.

As an additional, non-limiting example of a linear model, a KNeighbors Regressor (KNN) may be used. K-Nearest Neighbors (KNN) classifies a data point based on its nearest neighbors in a graph. Kramer, O.: K-nearest neighbors. In: Dimensionality reduction with unsupervisednearest neighbors, Springer: 13-23 (2013). This algorithm is a nonparametric supervised learning method used for classification and regression. In regression cases, the model takes the output value from a specific number of its nearest neighbors in the data, averages those values, and outputs that average. This algorithm does not make assumptions, so it may handle outliers and minimize error better than decision trees and linear regression. This model represents a novel approach to ensemble learning and is capable of high training speeds and high performance.

As an additional, non-limiting example of a linear model, an Elastic Net Regressor (ENR) may be used. Elastic Net is a regularized regression model that combines 11 and 12 penalties. This may be described as using lasso and ridge regression. Verma, Y.: Hands-on tutorial on elasticnet regression (2021). By combining both penalties, ENR dramatically reduces overfitting. This model also performs feature selection, removing unnecessary features from the data. ENR represents a novel use of penalties and feature selection.

Data captured using main bodies of the present disclosure must be prepared prior to processing via pre-processing. The image data set is converted into arrays with preprocessing and stored in a list format with assigned labels. Images are appended to a single data array with a corresponding label array. Data augmentation techniques including cropping, zooming, height and width shift, and horizontal flipping can be used, as well.

The present disclosure relates to cloud integration for real-time measuring. The glucose level estimation obtained from an ANN model is sent to the Cloud using Hypertext Transfer Protocol Secure (HTTPS). Additionally, a cloud-based database in the cloud is configured to store the glucose concentration data from the ANN model. An exemplary database for use is InfluxDB, which is written in the Go programming language for storing and retrieving time series data in fields such as operations monitoring, application metrics, Internet of Things sensor data, and real-time analytics. A database must be flexible enough to store data from each subject separately using tags. The integration with the Cloud is done using the Raspberry Pi, which is connected in Real-Time. The values that arrives to the database are immediately read by a mobile application for informing a subject. An overall workflow of the system of the present disclosure can be seen in FIG. 8.

The present disclosure relates to a mobile application to display real-time results, as described above. The application is capable of displaying at least current values, historical values, and statistics relating to glucose levels. Within the application, users can review their current glucose measure and also view a chart of their previous measures, allowing them to track glucose variation over a specific period of time. The application is connected to the database and provides continuous glucose monitoring and history data for users. The application further is enabled to pair with smart voice assistants. Such assistants are capable of replying to commands related to the current blood glucose concentration and recent history of blood glucose patterns. Assistants may be connected to a mobile application or device using various methods of interfacing including but not limited to by python script.

As a non-limiting example, a smart voice assistant may be connected as follows. First, two primary connections are established including one from a smart voice assistant to a python script in the middleware and a second from this script to a database. To set up the connection between the smart voice assistant and python script, intents and utterances are constructed in a developer console to enable the smart voice assistant to accept specific requests from the user by voice. After the successful setup in the developer console, the developer console is connected to the python script using Ngrok and Flask-ask. Next, in the python script, functions are implemented corresponding to each built intent including but not limited to retrieve current, maximum, or minimum values, and enter data into the database. Then a connection is established between the python script and the database that returns the value to the voice assistant for reproducing the voice with the glucose value.

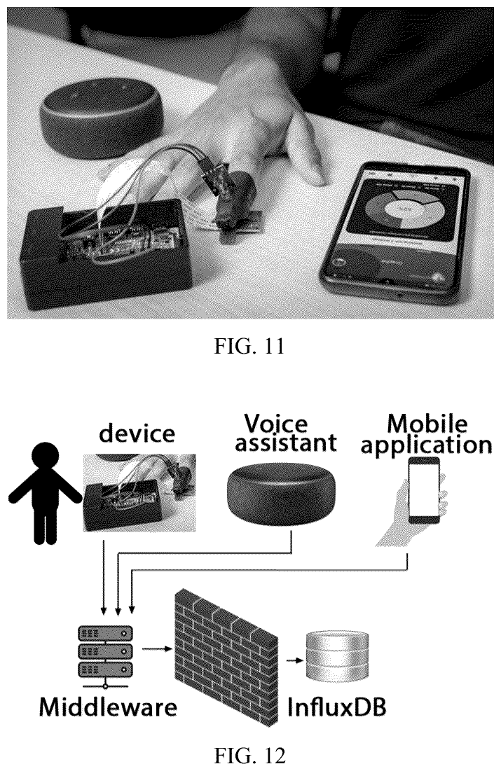

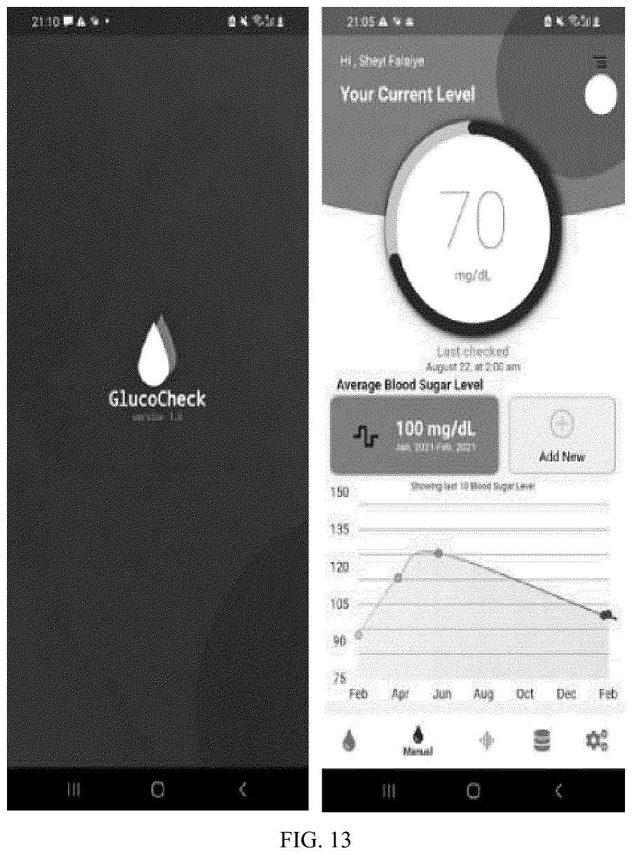

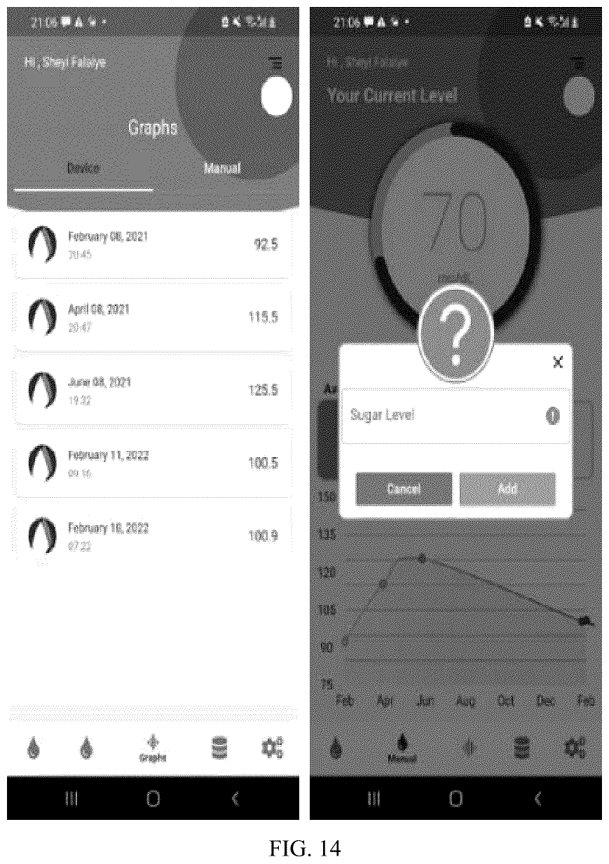

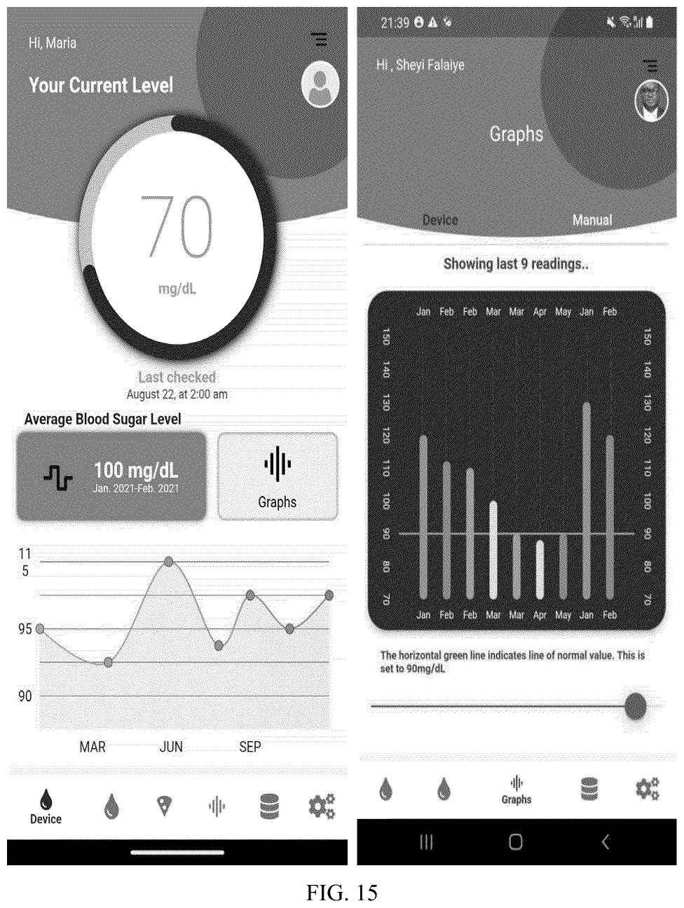

FIG. 11 shows a prototype compared with other pervasive devices for glucose estimation. FIG. 12 illustrates the architectural integration of the proposed device with other pervasive devices. Note that developed a middleware to ensure the confidentiality of the data in the database server. Users are able to review their current glucose measure, as well as a chart of their previous measures, illustrating glucose variation over a specific period of time. FIG. 13 shows the initial screen after the user opens the application (left) and the reading and graphs from the proposed prototype (right). The mobile application also allows users to manually enter readings from a glucometer for comparison purposes. This functionality provides additional training for the neural network according to the present disclosure. FIG. 14 shows the list of glucose reading (left) and the manual entry from glucometer (right). FIG. 15 shows additional example screens of the mobile app. In addition to the real-time line series graph shown in FIG. 15, the application is also able to show the average reading per month, weeks, days, hours. This can be download or directly sent to a healthcare provider via email.

The present disclosure relates to a non-invasive glucose monitoring system that leverages the computation power of IoT devices and can be used for diabetes management. The system disclosed does not require blood samples and is based on images taken from the finger or ear. The system provides an integrated end-to-end framework that helps with continuous blood glucose monitoring and, at the same time, provides a management system for daily-life decision support. Such support may include but is not limited to providing a user information to decide when to increase or decrease blood sugar (via food consumption, sugar consumption, beverage consumption, exercise, etc.). In addition, the framework is capable of accurately predicting blood glucose behavior regardless of factors like age, race, and physical condition. Compared with other current approaches, the presently disclosed system can be attached to additional extremities (e.g. ear) and still obtain reliable results. An ANN model is used to classify and estimate the blood glucose concentration from the images. Images taken from both the ear and fingers of subjects display acceptable accuracy.

Advantages of devices, systems, and methods described herein are many. As non-limiting examples, the present disclosure eliminates the need for invasive blood sampling, providing a painless and convenient alternative for glucose monitoring. The present disclosure offers devices, systems, and methods for improved accuracy and reliability by leveraging machine learning algorithms and analyzing spectroscopy images. Systems and devices described herein include compact and portable designs. Such designs enable users to monitor glucose levels anytime and anywhere. Furthermore, the user-friendly interfaces described herein make non-invasive glucose monitoring accessible to individuals of varying technical expertise, empowering them to manage their diabetes effectively.

Devices, systems, and methods described herein represent significant advancements in non-invasive blood glucose estimation. The present disclosure offers reliable, convenient, and accurate solutions for routine glucose monitoring. As a non-limiting example, one or more combinations of spectroscopy, image processing, and machine learning techniques establish a new standard in the field and hold great potential to enhance the quality of life for individuals with diabetes.

### EXAMPLES

### Example 1

Methods:

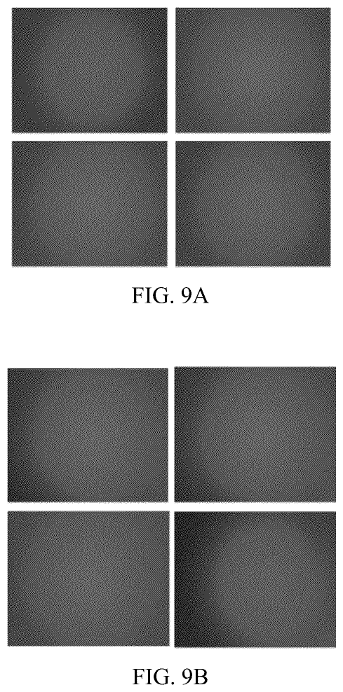

The monitoring system used Raspberry Pi (RPi), a portable camera (RPi camera), and a visible light laser. The RPi camera captured sets of images when visible light laser passed through skin tissue. The RPi camera captures one image every eight seconds over two minutes, for a total of 15 images. Brightness and contrast levels are set to 70 cycles/degree, camera ISO sensitivity is set to 800 and resolution is set to 640*480. FIGS. 5A-5B and 5C show the device attached to the finger and ear respectively. The glucose concentration was estimated by an ANN (Artificial Neural Network) model, as shown in FIG. 6, using the absorption and scattering of light in the tissue within the images. The image data set was converted into arrays with preprocessing and stored in a list format with assigned labels. Images, as shown in FIGS. 9A-9B, were then appended to a single data array with a corresponding label array. Data augmentation techniques including cropping, zooming, height and width shift, and horizontal flipping were used to train the model. This was developed using TensorFlow, Keras, and Python code. The data was then passed to the ANN model for glucose estimation.

Data were collected from 8 individuals under IRB approval at Kennesaw State University (IRB-FY22-318). Two different datasets were collected using the system. The datasets consisted of 7 subjects that provided images from their ears, and 8 volunteers that provided images from their fingers. About 80% of the data was used for training the model, as described herein, and about 20% of the data was used for testing. The procedure that was followed for data acquisition involved 2 steps. First, a finger prick test was performed to get the blood glucose reading using a commercially available Glucometer (FORA 6 Connect BG50 Blood Glucose Starter Testing (FOR A, “FORA 6 Connect BG50KT50 Blood Glucose”, (2020)). The system then captured 15 images each of finger and ear.

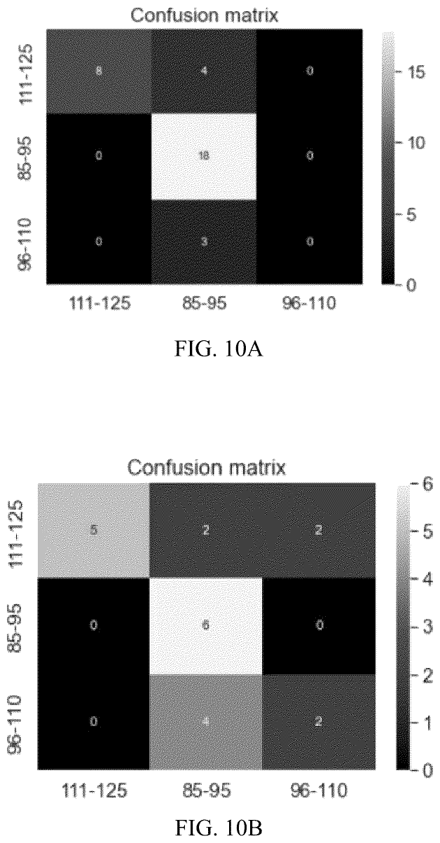

Starting with data preparation, Labelbinarizer module of the Python library sklearn was used to convert the image data to the needed binary image format and store it into an array associated with its corresponding labels/categories (85-95 mg/dL, 96-110 mg/dL, 111-125 mg/dL). The next step was data augmentation, which provided the amount of data needed to train and test the model. Data augmentation is a method known in the art and includes but is not limited to cropping, zooming, height and width shift, and horizontal flipping. Finger and ear datasets were not mixed to check how each model performed individually. Hence, two models were built, one for the finger dataset and the second one for the ear dataset. The models estimated the blood glucose concentration for each place, finger and ear. For evaluation purposes, classification reports, accuracy, and a confusion matrix were used to assess the model. Accuracy of the system was assessed by comparing the glucose readings from the system with readings from a glucometer. A confusion matrix was used to show the classification accuracy. The x-axis and y-axis showed labels of the blood glucose value; in this case, 111-125, 85-95, and 96-110, as shown in FIG. 10A for the finger readings.

Results:

FIG. 9A shows four images taken from one participant in the finger data collection. The images were taken in seconds 8 (top left), 16 (top right), 24 (bottom left), and 32 (bottom right). Images were taken after the finger prick. FIG. 9B shows four images that were taken from one of the earlobes of the participants in seconds 8 (top left), 16 (top right), 24 (bottom left), and 32 (bottom right).

Initial results using limited data provided an accuracy rate of about 79% for finger readings, as shown in a confusion matrix in FIG. 10A and also Table 2, which displays how many images were correctly and incorrectly classified. FIGS. 10A-10B include x-axes referring to correct estimates, while the y-axes show incorrect estimates. The unit for all x and y values is mg/dL. As shown in FIG. 10A, the ANN model classified 8 images correctly and 4 images incorrectly in the 111-125 mg/dL category. For the 85-95 mg/dL category, 18 images were correctly classified and 0 images were classified incorrectly. All 3 images in the 96-110 mg/dL category were incorrectly classified. This poor level of accuracy is due to the limited data set for these values.

A confusion matrix for ear readings showed an accuracy rate of about 62%, as seen in FIG. The model classified 5 images correctly and 4 images incorrectly in the 111-125 mg/dL category. In addition, 6 images were correctly classified and 0 images were classified incorrectly in the 85-95 mg/dL category. Finally, 2 images were correctly classified and 4 images were incorrectly classified in the 96-110 mg/dL category.

The results showed that optical techniques and machine learning methodologies can effectively measure blood glucose when the light is transmitted through the skin tissue and absorbed post-transmission. Overall, the system described herein exhibited an acceptable accuracy compared to other studies, as shown in Table 2. Of these other studies, the presently disclosed system is the only study to have shown efficacy with the human ear.

TABLE 2
Result Accuracy Comparison

| | | | No. | | | Mobile | |
|---|---|---|---|---|---|---|---|
| Work | Body Part | Technique | Subjects | Accuracy | Real-time | Application | Year |
| Present | Finger | Binary format of | 8 | 79% | Yes | Yes | 2022 |
| Disclosure | and Ear | image + CNN | | | | | |
| [29] | Finger | Infrared-multivariate | 3 | N/A | No | No | 1992 |
| | | calibration model | | | | | |
| [1] | Finger | Histogram and ANN | 514 | 90% | No | Yes | 2019 |
| [13] | Oral | Attenuate Total | 131/414 | 86.3% | No | No | 2018 |
| | mucosa | Reflection and | | | | | |
| | | hollow fibers | | | | | |
| [27] | Finger and | Reflected Optical | 12 | Correlation 0.86 | No | No | 2019 |
| | Wrist | Signal | | | | | |
| [19] | Forearm | Spectra Analysis of | 1 | 87.5% | No | No | 2003 |
| | | tissue light path | | | | | |

[29]: Robinson, M. R., et al., Clinical Chemistry 1618-1622 (1992)
[1]: Alarcón-Paredes, A., et al., “An IoT-based non-invasive glucose level monitoring system using raspberry pi”, Applied Sciences 3046 (2019)
[13]: Kasahara, R., et al., Biomedical optics express, 289-302 (2018)
[27]: Rachim, V. P., et al., Sensors and Actuators B: Chemical 173-180 (2019)
[19]: Maruo, K., et al., IEEE Journal of selected topics in quantum electronics, 322-330(2003)

### Example 2

Methods:

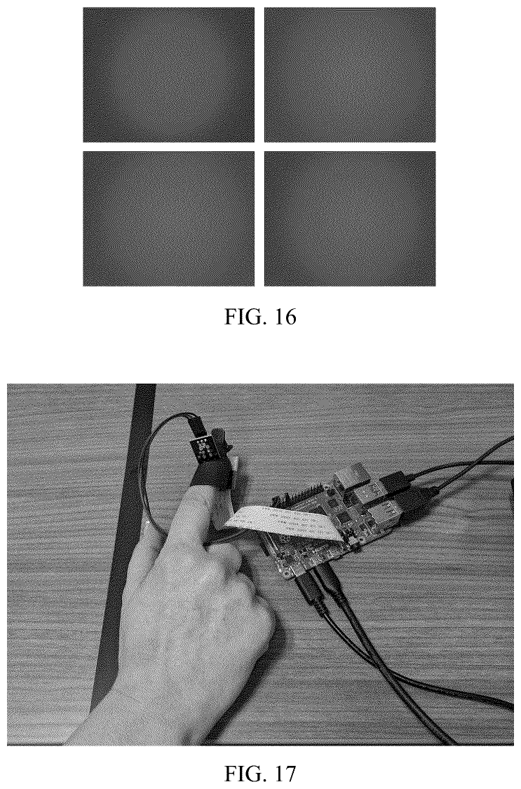

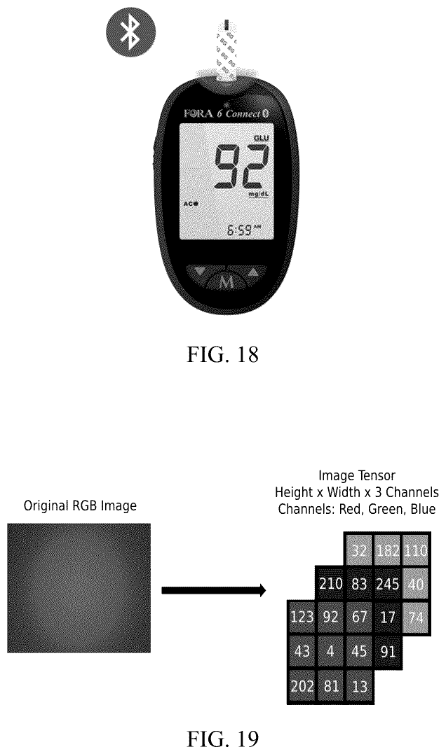

Example 2 used the results of Example 1 described above. Images were chosen instead of other forms of spectroscopy measurement, such as light intensity and PPG signals, because image capture is more replicable, accessible, and faster than other methods of spectroscopy data collection. Spectroscopy images were collected from the fingers of 43 participants between 18-65 years old. The demographic of the participants were 23 females and 20 males, aged between 18 and 65 years, and with ethnic/racial self-identification as 7 white, 7 black, 13 Latino, and 16 Asian individuals. Two sets of 15 images were collected per participant. The first set was collected in a low-glucose fasting state. The second set was collected one hour following a meal. Blood glucose was determined via finger prick using a commercial glucometer (FORA 6 Connect BG50) per manufacturer instructions. A set of 4 images is presented in FIG. 16. The images were taken after the finger prick at seconds 8 (top left), 16 (top right), 24 (bottom left), and 32 (bottom right). All images were collected from fingertips in the same format. A 640×480 resolution was chosen to preserve small details without sacrificing computing time and resources. The standard RGB color format was used. After removing any unclear images, the final dataset consisted of 1128 samples, each with two features, the image, and the corresponding blood glucose value. FIG. 17 shows an image of a participant during the data collection process, while FIG. 18 shows the glucometer device used for comparison purposes.

Data Set Creation

Data transformation techniques were applied to the original data to generate three datasets, as described below.

An Image Tensor Dataset was prepared. The “Tensor Dataset” was created in order to train the CNN models (VGG16 and MobileNetV2). Tensors are multi-dimensional matrices of numbers used in linear algebra. Tensor application extends to images since images are multi-dimensional matrices of numbers as well. An image matrix consists of at least three dimensions including but not limited to height, width, and color (including but not limited to red, green, and blue).

To convert an image into a tensor, a three-dimensional matrix (tensor) was created with the resolution of the image and the color format. Since images used herein were 640×480 pixels using the RGB color format, the image tensor was also 640×480 pixels by three colors. Then each color value for each pixel was entered into each value in the tensor, obtaining a tensor of 921,600 values. The resulting image tensor dataset was maintained at 160×120×3 pixels to decrease computational time and necessary resources, when compared to a 640×480 pixels dataset. The final dataset included the tensors with their corresponding blood glucose value(s). A visual demonstration of the image-tensor conversion can be seen in FIG. 19.

CNN models can be trained with such tensors because they use filtering techniques to analyze and process them. These filtering techniques may not be available in other machine learning algorithms. This may make the two CNN models MobileNetV2 and VGG16 advantageous.

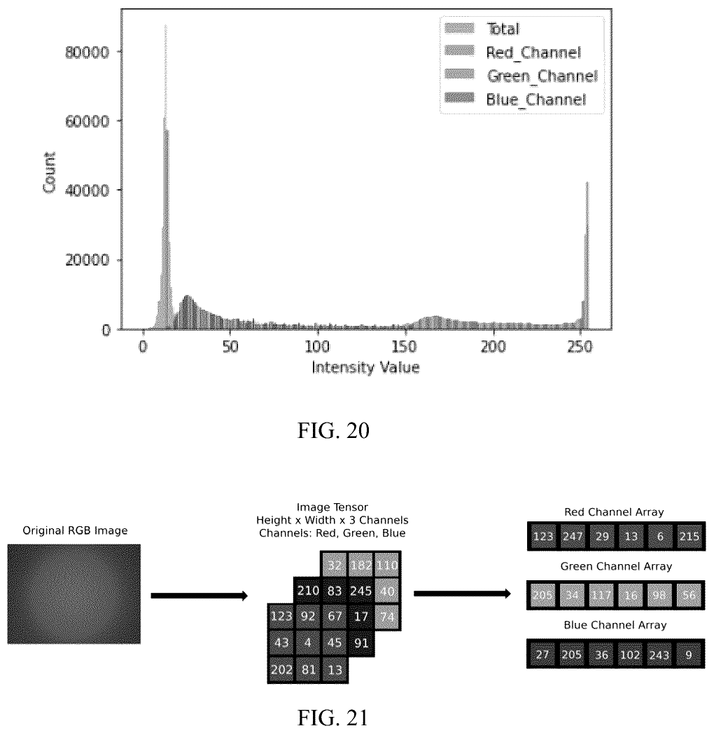

Color Intensity Datasets were prepared. Four datasets were created based on extracting color intensity from original images. For each possible value of red, green, and blue (0-255), the number of pixels with that same value in an image can be counted and recorded in a histogram. Guzm′an-Guzm′an, I.P., et al., An iot-based noninvasive glucose level monitoring system using raspberry pi (2019). Through this process, a histogram with RGB values on the x-axis (256 possible values for red, green, and blue) and the number of pixels on the y-axis can be created, as shown in FIG. 20. This process of counting pixel-intensity values for each color was used to create three datasets: “Red Intensity”, “Blue Intensity,” and “Green Intensity.” Each dataset consisted of 257 features including 256 features for each possible value of that color and one feature for the blood glucose value of that image. Lastly, a final dataset, named “RGB-Intensity”, was created by combining the intensity values for all three-color channels. The RGB-Intensity dataset consisted of 769 features including 256 values of red, 256 values of green, 256 values of blue, and one value for blood glucose. Guzm′an-Guzm′an, I.P., et al., An iot-based noninvasive glucose level monitoring system using raspberry pi (2019).

Image Measurement Datasets were created. Five datasets were created by extracting measurement data from the images. To create the dataset, each image in the dataset was split into four channels including red, green, blue, and grayscale (the image with color removed). Then, for each color channel, the channel's pixel center of mass, minimum, maximum, mean, median, standard deviation, and variance were calculated. To calculate these values, the images were first converted into numerical tensors, then their tensors (3-dimensional matrix) were converted into an array for each channel, and then each channel array (1-dimensional list) was used for calculations such as mean, median, minimum, maximum, and the like. A demonstration of this process can be seen in FIG. 21.

Values for each channel were compiled into the same dataset with the correct blood glucose value and repeated for every image. The resulting “Measurement Dataset”, consisted of 29 features: seven measurements for each of the four channels and one feature for the blood glucose. After the creation of this dataset, four new datasets were created by merging the measurement features of each image with the intensity values of the same image created in the previously mentioned intensity datasets. This process resulted in four new datasets including “Red-Measurement”, “Green-Measurement”, “Blue-Measurement”, and “RGBMeasurement.” The first three new datasets contained 285 features including 256 for the pixel intensities, 28 for the measurement features, and one for the blood glucose value. The last new dataset contained 797 features including 256 for each color channel, 28 measurement features, and one for the blood glucose value.

Model Training, Tuning, Testing, and the Like

After creating the datasets, each model was trained, tuned, and tested to each dataset to compare results, with only two exceptions. Since they can only be trained on tensor data, VGG16 and MobileNetV2 were only trained on the Tensor Dataset. Furthermore, the other linear models could only be trained on scalar data, so they were trained on every dataset except for the Tensor Dataset. The CNN models were trained using image data generators, which come with the TensorFlow library for Python that was used for training models. Moreover, before the training process, the image data generators were used to scale down the pixel values from 0-255 to 0-1 to reduce error and GPU usage. Besides these changes during training, the testing of CNN models was the same as the other models. Additionally, since the AdaBoost Ensemble Learning algorithm used another algorithm as a base estimator, for each dataset, the AdaBoost model was trained with the model that had the highest accuracy for that dataset. A summary of the models trained with each dataset can be seen in Table 2A.

TABLE 2A
Models Trained on Each Dataset.

| Tensor | Intensity | Measurement | Intensity- |
|---|---|---|---|
| Dataset | Dataset | Dataset | Measurement Dataset |
| VGG16 | Random Forest | Random Forest | Random Forest |
| MobileNetV2 | Support Vector | Support Vector | Support Vector |
| | Bayesian Ridge | Bayesian Ridge | Bayesian Ridge |
| | XGBoost | XGBoost | XGBoost |
| | HGB | HGB | HGB |
| | AdaBoost | AdaBoost | AdaBoost |
| | KNeighbors | KNeighbors | KNeighbors |
| | Elastic Net | Elastic Net | Elastic Net |

To train the models, all of the datasets were split into training/testing splits where the training data was used to fit the model, and the testing data was used to measure the model's performance. The training/testing split ratio was 75:25 to ensure sufficient data to train the models and to ensure that no overfitting. After creating training/testing splits, each model in the set was fitted to training data and then tested. To ensure that the models were compared effectively, each model's hyper-parameters were tuned to each specific dataset to minimize error and overfitting. After the models were finished with training and tuning, they were tested, and the results were recorded in a table.

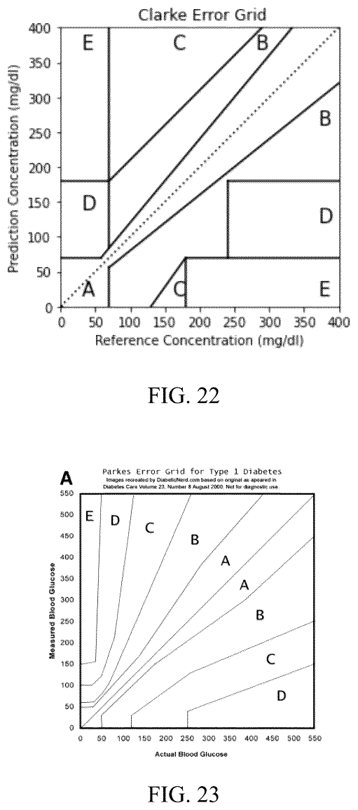

Three distinct metrics were considered for testing/tuning the models including mean absolute error (MAE), root mean squared error (RMSE), and the Clarke Error Grid. The MAE is the mean of all errors between the blood glucose values that a model predicts and the actual blood glucose value tied to an image. The RMSE is the root of the mean of each error squared. MAE may be advantageous as a more direct metric for calculating error, as it is unbiased towards all errors and treated as an average. Because it squares the errors, RMSE may be advantageous as being biased against large prediction errors, making it weighted against outliers. RMSE may be used in scenarios when an increase in error is disproportionate to the effect. As a non-limiting example, if the error increases from 5 to 10 and the effect is four times as bad, RMSE may be used. Since RMSE is always higher or equal to MAE, the difference between the two values is useful for evaluating outliers. If RMSE is significantly higher than MAE, then there are outliers in the predictions. For this reason, RMSE was used to tune the models to reduce overfitting but not recorded in the results or evaluation. Lastly, Clarke Error Grids were used to evaluate models since they have been widely used for several decades to evaluate the performance of blood glucose meters. Clarke Error Grids are scatterplots with predicted blood glucose values on the y-axis and actual blood glucose values on the x-axis. The grid is split into several zones, and each zone signifies a level of risk of a negative outcome due to the measurement error in blood glucose values, as shown in FIG. 22. The five zones of FIG. 22 include A— Clinically Accurate, B—Clinically Acceptable, C—Overcorrection, D—Failure to Detect/Treat, and E—Erroneous Treatment. Clarke, W. L., et al., Evaluating clinical accuracy of systems for self-monitoring of blood glucose (1987). Additionally, Parkes error grids can be used. The Parkes error grid specifies five risk levels and has been differentiated with grids for diabetes type 1 and type 2. J. L. Parkes, et al., Diabetes Care, 23(8): 1143-1148 (2000). Patients with type 1 diabetes were not studied, and therefore the grid for type 2 was used, as shown in FIG. 23. MAE, RMSE, and the Clarke Error Grid were all used when measuring the performance of the models during training and testing.

Standards.

The ISO 15197 standard defines specifications for reliable medical devices, including glucometers. The ISO standard released in 2013 for glucose monitoring devices and systems for selftesting (15197:2013) has tighter accuracy requirements than the previous ISO standard set in 2003. The 2013 standard requires that 95% of the results are within a glucose concentration of pm15 mg/dL, compared with the reference method, for concentrations less than 100 mg/dL, or pm15% of zones A and B of the Parkes (Consensus) Error Grid for diabetes type 1.

Results:

For comparing the performance of the models, MAE and Clarke Error Grid (Zone A Percentage) metrics were used. The percentage of data points that fell into each zone of the clinical outcome were determined by analyzing the grid. Zone A's percentage was calculated by recording the number of predictions in Zone A (Clinically Accurate) as a percentage of the total number of predictions made. After the models were trained and tuned, they were tested with the testing data, and the results were recorded in Table 3 and Table 4 respectively.

TABLE 3
Model Testing Results from Tensor and Intensity Datasets -
MAE and Zone A percentages from clarke error grid analysis.

| | Image Tensor | Red-Intensity | Green-Intensity | Blue-Intensity | RGB-Intensity |
|---|---|---|---|---|---|
| | (IT) | (RI) | (GI) | (BI) | (RGBI) |
| VGG16 | 16.58-87.59% | — | — | — | — |
| MobileNetV2 | 15.68-87.23% | — | — | — | — |
| Random Forest | — | 13.17-86.17% | 13.31-85.11% | 14.04-86.17% | 12.46-88.65% |
| Elastic Net | — | 15.59-85.46% | 16.23-82.27% | 15.53-84.4% | 14.42-84.4% |
| KNeighbors | — | 9.88-90.78% | 14.06-88.3% | 14.35-85.46% | 10.84-88.65% |
| Support Vector | — | 14.43-89.36% | 15.71-89.36% | 14.3-89.36% | 13.14-88.65% |
| Bayesian Ridge | — | 15.43-85.11% | 16.01-83.33% | 15.34-83.33% | 14.28-84.4% |
| XGBoost | — | 12.93-87.94% | 14.1-84.75% | 13.97-84.75% | 12.26-89.72% |
| HGB | — | 13.12-86.88% | 14.99-84.04% | 14.37-83.69% | 12.53-87.59% |
| AdaBoost | — | 9.66-90.78% | 13.31-87.94% | 14.08-85.46% | 10.95-88.65% |

TABLE 4
Model Testing Results from Measurement Datasets - MAE
and Zone A percentages from clarke error grid analysis.

| | | Red- | Green- | Blue- | RGB- |
|---|---|---|---|---|---|
| | Measurement | Measurement | Measurement | Measurement | Measurement |
| | (ME) | (RM) | (GM) | (BM) | (RGBM) |
| Random Forest | 14.27-83.33% | 12.85-87.23% | 12.63-86.52% | 13.91-85.82% | 12.74-88.65% |
| Elastic Net | 16.38-81.56% | 15.68-84.04% | 16.89-85.11% | 15.55-81.56% | 14.41-83.69% |
| KNeighbors | 15.02-81.91% | 9.55-90.78% | 14.3-86.17% | 15.81-84.4% | 12.43-87.59% |
| Support Vector | 16.13-89.72% | 14.3-87.94% | 15.02-89.01% | 14.58-87.23% | 13.28-87.94% |
| Bayesian Ridge | 16.37-81.56% | 15.52-84.04% | 17.43-85.46% | 15.52-82.62% | 14.3-83.33% |
| XGBoost | 14.51-83.69% | 13.03-86.88% | 12.86-88.3% | 13.6-86.52% | 12.89-87.59% |
| HGB | 14.78-81.21% | 13.71-85.11% | 13.17-86.17% | 13.7-85.11% | 12.58-87.94% |
| AdaBoost | 15.13-80.5% | 9.4-90.78% | 12.74-86.88% | 13.41-87.59% | 13.18-86.52% |

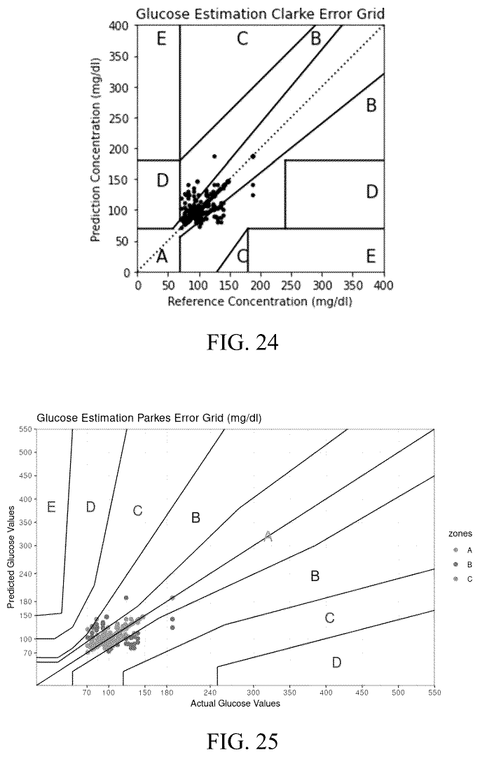

AdaBoost with KNeighbors trained on the Red-Measurement dataset provided the most accurate estimates of blood glucose among all of the dataset-models tested. This dataset-model combination had an MAE of 9.4 mg/dl, an RMSE of 16.72 mg/dl, and a Clarke Error Grid Zone A Percentage of 90.78% as illustrated in FIG. 24. The Parkes Error Grid was also used to further evaluate the model. FIG. 25 shows the results based on this error. In this case, 87.2% of the values fell into Zone A or “clinically accurate”, 11.7% fell in Zone B or “clinically acceptable”, and 1.1% fell in Zone C or “clinically inaccurate—likely to affect clinical outcome”.

Table 5 shows a sample of 15 individual participants and the comparison between the glucometer reading and the proposed approach estimations. The average deviation between the actual value and the estimated value was within ±1.02 mg/dL and ±4.5 mg/dL. Based on these results, the proposed prototype and model met the International Standards Organization (ISO) 15197 standard that requires ±15% of zones A and B.

TABLE 5
Sample Comparison of Results of the Proposed Approach and The Ground Truth

| | | | | Glucometer | | Proposed Approach | | Errors (mg/dL) | | |
|---|---|---|---|---|---|---|---|---|---|---|
| | Demographics | | | Readings | | Readings | | | | Ave. |
| | Age | Gender | Race | First | Second | First | Second | Error 1 | Error 2 | Error |
| 1 | 43 | Female | White | 83 | 85 | 83 | 88 | 0.0 | 3.53 | 1.77 |
| 2 | 21 | Male | Black | 113 | 120 | 113 | 115 | 0.0 | 4.17 | 2.09 |
| 3 | 24 | Female | White | 98 | 98 | 99 | 99 | 1.02 | 1.02 | 1.02 |
| 4 | 33 | Female | Latino | 92 | 95 | 93 | 96 | 1.09 | 1.05 | 1.07 |
| 5 | 24 | Male | Latino | 100 | 124 | 96 | 125 | 4.0 | 0.81 | 2.41 |
| 6 | 18 | Female | Asian | 82 | 84 | 83 | 87 | 1.22 | 3.19 | 2.21 |
| 7 | 18 | Female | Black | 95 | 100 | 98 | 94 | 3.16 | 6.0 | 4.58 |
| 8 | 31 | Female | White | 108 | 131 | 105 | 123 | 2.78 | 6.11 | 4.45 |
| 9 | 41 | Male | Latino | 84 | 105 | 89 | 102 | 5.95 | 2.86 | 4.41 |
| 10 | 24 | Female | Latino | 87 | 96 | 89 | 95 | 2.3 | 1.04 | 1.67 |
| 11 | 29 | Female | Black | 80 | 102 | 82 | 101 | 2.5 | 0.98 | 1.74 |
| 12 | 39 | Male | Asian | 80 | 102 | 82 | 101 | 2.5 | 0.98 | 1.74 |
| 13 | 27 | Female | White | 106 | 80 | 105 | 84 | 0.94 | 5.0 | 2.97 |
| 14 | 29 | Male | Asian | 83 | 101 | 85 | 99 | 2.41 | 1.98 | 2.2 |
| 15 | 28 | Male | Asian | 87 | 140 | 86 | 133 | 1.15 | 5.0 | 3.08 |

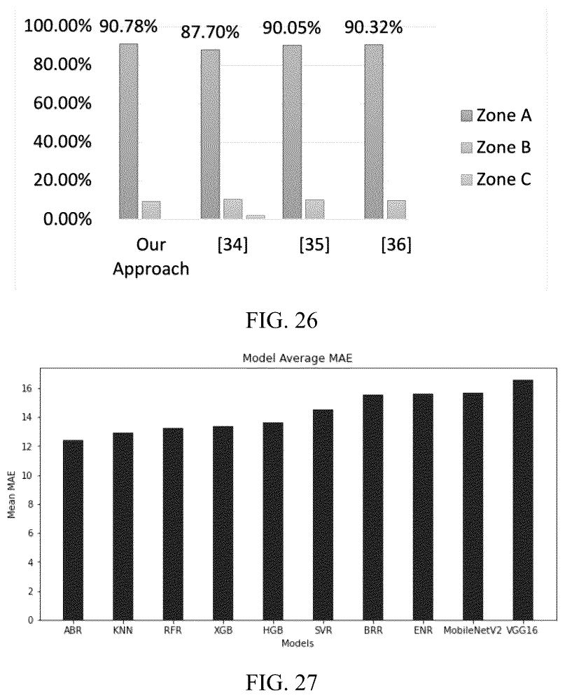

The Clark Error method was used to compare accuracy with studies of similar noninvasive approaches. FIG. 26 illustrates our accuracy compared with these approaches. FIG. 26 shows the following similar approaches: [34]: E. Monte-Moreno, Artificial Int. in Med., 53(2): 127-138 (2011); [35]: K. Yamakoshi, J. of Biomed. Opt., 11(5): 054028 (2006); and [36]: A. Alarcon-Paredes, Applied Sci., 9(15): 3046 (2019). However, none of these studies used the Parkes Error grid that penalized the error in clinical accuracy more and it is more difficult to accomplish. Therefore, we hypothesize that the accuracy of our approach is superior to others in the research field.

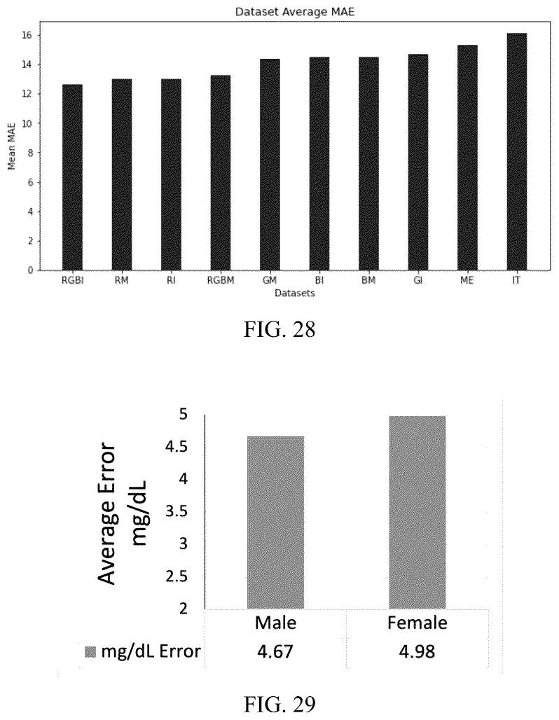

From best to worst, the models ranked AdaBoost, KNeighbors, Random Forest, XGBoost, HGB, Support Vector, Bayesian Ridge, Elastic Net, MobileNetV2, and VGG16 as displayed in FIG. 27. From best to worst, the datasets ranked RGB Intensity, Red Measurement, Red Intensity, RGB Measurement, Green Measurement, Blue Intensity, Blue Measurement, Green Intensity, Measurement, and Image Tensor as shown in FIG. 28. The datasets containing Red and RGB data outperformed the other datasets. Combining measurement and intensity values did not seem to improve performance for the red dataset, but instead hindered it. Datasets with Blue and Green data appeared to perform equally, but their performance was inferior to the Red and the combined RGB overall. The Green data, but not the Blue, seemed to perform better after combining intensity and measurement data.

The intensity datasets performed better than the measurement datasets, and the dataset with only measurement values performed significantly worse. The image tensor dataset performed the worst of all datasets, while the CNN models performed the worst among the group of models. AdaBoost and KNeighbors performed the best with every dataset they were trained on, while XGBoost, Random Forest, and HGB generally outperformed the other models. These results suggested that the best data for blood glucose estimation by spectroscopy may be color intensity data focused on either the red channel or all three channels, though other data still provides value. The results further suggest that the KNeighbors algorithm is well-suited for blood glucose estimation with scalar data and using AdaBoost as an ensemble learner can boost performance. Models that used boosting and bagging (XGBoost, AdaBoost, HGB, etc.) outperformed models that did not (Elastic Net, Bayesian Ridge, Support Vector). Furthermore, the penalties and feature selection in Elastic Net and the binning in HistogramBased Gradient Boosting did not seem to increase performance compared to bagging and boosting. Finally, both the dataset and model results suggested that Convolutional Neural Networks and Tensor datasets performed worse than Linear Models, Ensemble Learners, and Scalar Data.

From training, tuning, and testing ten machine learning models on ten different datasets, it was determined that a particularly advantageous model for estimating blood glucose through spectroscopy images was AdaBoost trained with KNeighbors. Color intensity data collected from the red channel represented an advantageous image data for training a model. The highest performing dataset and model recorded a final Mean Absolute Error of 9.4, a Root Mean Squared Error of 16.72, and a Clark Error Grid Zone A Percentage of 90.78%. Intensity data outperformed measurement and tensor data, while the red and RGB channels outperformed all other color channels. Models that utilized bagging and boosting outperformed those which did not, while linear models outperformed CNN models, regardless of their support for bagging or boosting.

### Example 3

Results:

The accuracy of the device was tested based on differences in race and ethnicity, age, and gender per methods defined above. No significant difference in mean glucose estimates were observed between males and females (FIG. 29). Nail polish may have had an impact on light absorption, and therefore participants were asked to avoid using dark colors nail polish.

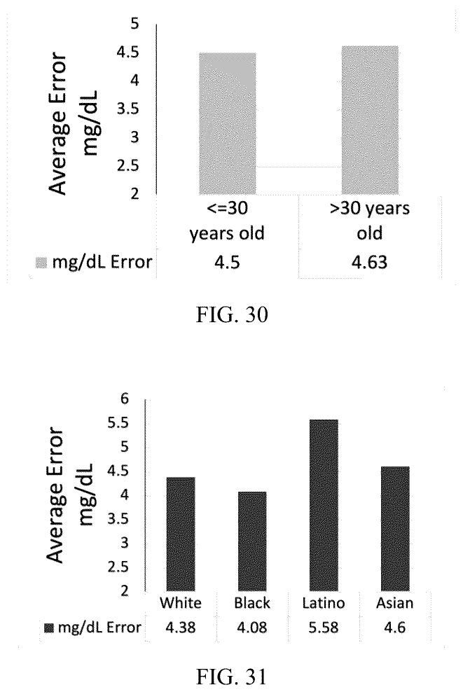

No difference was observed in mean glucose concentration when data was stratified by age. FIG. 30 shows average error of individuals divided as above or below the mean age (30 years). No significant difference was found in the average error between both groups.

Differences in skin color and pigmentation were tested. In FIG. 31, the results of the average error in terms on race are shown. Glucose estimates for Latino and Asian participants were less accurate than those for the other participants, while estimates for black individuals were more accurate than those for white participants.

That which is claimed is:

1. A non-invasive blood glucose monitoring system, the system comprising:
a portable main body configured to be secured adjacent a portion of a user, wherein the portion of a user is an ear or a finger;
a plurality of sensors disposed in the main body and configured to collect information relating to user characteristics via the portion of the user, wherein the plurality of sensors comprise at least a light source and a camera wherein the light source emits light that permeates the portion of the user and produces conditions for an image to be captured by the camera through infrared spectroscopy, and wherein the information comprises at least the image; and
a computing device configured to receive the information relating to the user characteristics in real-time, to analyze, based on a neural network model, a blood glucose level of the user, and to output a blood glucose level estimation derived from the model, wherein the blood glucose level estimation is based at least on a correlation between one or more extracted features in the image and a blood glucose concentration value.

2. A non-invasive blood glucose monitoring system, the system comprising:
a portable main body configured to be secured adjacent a portion of a user, wherein the portion of a user is an ear or a finger;
one or more sensors disposed in the main body and configured to collect information relating to user characteristics via the portion of the user, wherein the one or more sensors is configured for infrared spectroscopy; and
a computing device configured to receive the information relating to the user characteristics in real-time, to analyze, based on a neural network model, a blood glucose level of the user, and to output a blood glucose level estimation derived from the model, wherein the blood glucose level estimation is based at least on a correlation between one or more extracted features in the image and a blood glucose concentration value.

3. A non-invasive blood glucose monitoring system, the system comprising:
a portable main body configured to be secured adjacent a portion of a user, wherein the portion of a user is an ear or a finger;
one or more sensors disposed in the main body and configured to collect information relating to user characteristics via the portion of the user, wherein the one or more sensors is configured for infrared spectroscopy; and
a computing device configured to receive the information relating to the user characteristics and to analyze, based on a model, a blood glucose level of the user.

4. The blood glucose monitoring system of claim 3, wherein the main body comprises a first portion and second portion coupled together.

5. The blood glucose monitoring system of claim 3, wherein the one or more sensors comprise a laser and a camera.

6. The blood glucose monitoring system of claim 5, wherein the laser permeates the portion of the user and produces an image that is captured by the camera to collect the information through infrared spectroscopy.

7. The blood glucose monitoring system of claim 3, wherein the collected information comprises information indicative of blood glucose levels, blood glucose level estimations, glucose levels, glucose level estimations, or a combination thereof.

8. The blood glucose monitoring system of claim 3, wherein the computing device is functionally disposed to allow operations of the model to produce an output.

9. The blood glucose monitoring system of claim 8, wherein the output is a blood glucose level estimation.

10. The blood glucose monitoring system of claim 9, wherein the estimation is at least 79% accurate.

11. The blood glucose monitoring system of claim 9, wherein the estimation is at least 62% accurate.

12. The blood glucose monitoring system of claim 3, wherein the model is a neural network.

13. The blood glucose monitoring system of claim 12, wherein the information is pre-processed before being sent to the neural network.

14. The blood glucose monitoring system of claim 12, wherein the neural network is selected from a group consisting of a convolutional neural network (CNN) and an Artificial Neural Network (ANN).

15. The blood glucose monitoring system of claim 3, wherein the model is trained using about 80% of the information collected by the system and tested using about 20% of the information collected by the system.

16. The blood glucose monitoring system of claim 3, wherein the system further comprises a cloud-based database configured to store output from the model.

17. The blood glucose monitoring system of claim 16, wherein the system further comprises a mobile application functionally disposed to display outputs stored in the cloud-based database.

18. The blood glucose monitoring system of claim 17, wherein the mobile application provides continuous glucose monitoring and history data for users.

19. The blood glucose monitoring system of claim 17, wherein the mobile application allows users to manually enter glucometer readings for comparison purposes and for training the model.
